(19) United States
(12) Patent Application Publication
(10) Pub. No.: US 2017/0099857 A1
(43) Pub. Date: Apr. 13, 2017

(54) DRYING PROCESS FOR AGRICULTURAL FEEDSTUFFS

(71) Applicant: Agri-King, Inc., Fulton, IL (US)

(72) Inventors: Kristi Lynn Balk, Morrison, IL (US); Jeffrey Glen Horst, Clinton, IA (US)

(73) Assignee: Agri-King, Inc., Fulton, IL (US)

(21) Appl. No.: 14/881,396

(22) Filed: Oct. 13, 2015

(51) Int. Cl. F26B 11/02; G01N 33/02

(52) U.S. Cl. CPC A23K 3/005; G01N 33/02

(57) ABSTRACT

A method of drying feedstuff samples without substantially altering their composition is provided. The method includes placing feedstuff samples in one or more porous enclosures, such as bags. The enclosures allow for airflow to pass through them to the samples within without allowing said samples to escape. Enclosures holding the feedstuff samples are placed in a dryer. Multiple porous enclosures may be placed in the dryer concurrently. The dryer then subjects the feedstuff samples in the enclosures to heated airflow and rotational movement/tumbling to adjust the moisture content of the feedstuff samples. The heated air of the dryer has a temperature and airflow rate of at least 50 degrees Celsius and 500 CFM, respectively. Moreover, the rotational movement within the dryer has a rate of at least 40 RPM. The resulting dried samples have approximately 10% or less moisture remaining after 45 to 180 minuses in the dryer.

### FIELD OF THE INVENTION

The present invention relates generally to the removal of moisture from agricultural feedstuffs such as grams, forages and other byproducts. More specifically, the present invention relates to methods of drying feedstuff to reduce required drying time to approximately 45-180 minutes while the analytical composition of said feedstuffs remains substantially unchanged.

### BACKGROUND

Laboratories are constantly evaluating ways to discover faster turnaround times for preparing feedstuff and foodstuff samples for testing and/or other client needs. Large farms are feeding enormous volumes of feedstuffs to animals; thus placing a premium on quick feedstuff analysis for prompt nutritional balancing. Typically, wet feedstuffs and other forage contain a moisture level of 50% or more, whereas the normal final moisture level suitable for testing in feedstuffs is below 10%. Current and previous methods for drying are excessively long and/or alter the analytical composition of the original materials thus making them ill-suited for the current fast-paced market.

Previously utilized methods include conventional oven drying, microwave drying, hydration drying and/or vortex drying. Conventional oven drying typically involves forced air convection heat applied at approximately 60 degrees Celsius. With conventional oven drying, wet forage and other feedstuffs are commonly placed into metal or paper containers and are baked for 12-24 hours per half pound of wet forage.

Microwave drying can be completed in under 10 minutes for smaller samples of wet forage, however the technique is documented to have adverse effects on subsequent tests due to changes in the analytical composition of the samples. These compositional changes occur due to Maillard reactions and caramelization from pyrolysis at temperatures around 140 to 165 degrees Celsius. Additionally, the resulting dry forage from microwave drying is highly dependent on the operator. This is due to the inconsistent heating inside a microwave which are commonly referred to as “hot spots.”

Previous methods have incorporated drying processes into feedstuff sample preparation. However, these previous drying methods have drawbacks. In particular, these methods are unsuccessful when attempting to maintain the original analytical composition of the samples and/or are ineffective in substantially reducing the drying time needed. Oftentimes, these analytical composition changes are averse to the sample drying process as the samples are no longer representative of the feedstuff they were originality intended to represent. Additionally, the drying methods utilized are typically time-intensive and increase the turnaround time for testing samples.

In one example, U.S. Pat. No. 5,370,007 discloses a process for fiber analysis. The invention described therein relates to a method of conducting fiber analysis such as for determining the nutritional availability of forage and other feedstuffs. In the described method, the sample of feedstuff is placed in a bag of predetermined porosity. The closed bags are then placed in a container of heated detergent solution to remove all of the soluble solids from the feedstuff while retaining the fiber within the bag. The bags are then removed from the detergent and rinsed in hot water. Following the rinse, the bags are cleaned with an organic solvent, rinsed again, dried and weighed to determine the fiber content of the feedstuffs. The drying process is accomplished utilizing an oven.

In another example, U.S. Pat. No. 6,479,295 discloses a method for determining crude fat levels in feed, food and other materials utilizing filter media encapulation. In the method, the sample is encapsulated in filter media with the capability of retaining four microns size and larger particles while permitting flow of solvent through the filter media to extract crude fat. Specifically, the fat is quantitatively extracted from the filter chamber while all other components are retained in the filter chamber. The weight loss of the sample represents the fat content. Methods for drying of the samples is disclosed as evaporation and drying in an oven.

Another example, International Reference No. WO 99/02959 covers a container for use to find fiber content of foodstuff. The container described allows constituents of a sample to be removed in solution while leaving insoluble residue behind. The container is preferably rigid but may be made of non-rigid material as well. Additionally, the container is destroyed in the last step of the process. Therefore, the container is not reusable. The disclosed methods of drying the sample include evaporation and oven drying.

Another reference, international Reference No. WO 13/009062 discloses a dryer for agricultural and marine products. The disclosed dryer is similar to a drying rack with a frame and mesh or fabric spread across the frame to receive the agricultural and/or marine products. The preferred method of drying is via direct sunlight. The frame may also include electricity for radiant heat if sunlight is unavailable.

None of the above methods provides an efficient means for drying feedstuff samples. In addition, the above-described methods fail to result in substantially reduced drying times, particularly with respect to large feedstuff samples and/or multiple containers of feedstuff samples. Moreover, the above methods cannot be as easily integrated into feedstuff sample production facilities as the method of the present invention given the generally larger size, sometimes in excess of 209 cubic feet, of previously utilized methods.

Accordingly, there exists a need in the art for a method to substantially reduce drying time for feedstuff samples. The method should allow for quick drying of samples without altering the analytical composition of the sample. Furthermore, the method should also allow for the drying of multiple containers of feedstuff samples at the same time. Such a method should be easily integrated into already established feedstuff sample preparation facilities.

### SUMMARY

The present invention provides a method for fast drying large volumes of feedstuff samples utilizing a tumbling, forced air and heated drying source. The typical time for adequate drying utilizing the process of the present invention is reduced to 3 hours or less. This time generally represents a five to ten-fold decrease in the required drying time to prepare feedstuff samples when compared to previously utilized methods. A method of the present invention provides the optimum temperature to dry feedstuff samples while turning said samples and simultaneously forcing heated air through a container housing samples and through said samples within. The container of the preferred embodiment of the present invention is a bag that allows air to pass through but does not allow sample particles to pass through the pores of the bag due to the pores of the bag being sized smaller than the smallest sample particulates. The pores of the bag of the preferred embodiment are approximately 20 microns in size. The bag is also made of one or more materials that does not retain moisture within the material(s) itself. The materials may be one or more of the following: cotton, polyester, spandex, nylon, muslin, broad-weave, anti-static polyester, wood pulp and combinations thereof. The preferred embodiment of the bag also includes a zipper to open and close the bag to allow for the insertion, holding and removal of one or more feedstuff samples within. The bag may also include a retention mechanism for the zipper pull of the zipper to keep the zipper pull front tangling with other bags, other zipper pulls and/or hitting the interior surface of the drying apparatus of the preferred embodiment.

The drying apparatus in the preferred embodiment is a commercial grade tumbling dryer with an interior drum that provides rotational movement along a horizontal axis in order to tumble the contents within the dryer. Once the samples are dried to the required moisture content, at or below 10% in the preferred embodiment, the samples may be further processed, such as by grinding or pulverizing the samples, for testing.

In some embodiments, the method may include drying the feedstuff samples to a moisture content of approximately 10% or less. It is contemplated some samples may be tested at other moisture levels greater than 10% appropriate. The drying of the samples may occur in a dryer at a temperature of 40-220 degrees Celsius, drying in the preferred embodiment occurs at 60 degrees Celsius. The drying typically requires about 45 to 180 minutes in the preferred embodiment. More specifically, the preferred embodiment typically allows 40-50 bags of 230 gram samples of corn silage, with an initial moisture content of approximately 60-65%, to be dried to 10% or less moisture in approximately 150 minutes or less. Furthermore, the dryer may also rotate/tumble multiple containers holding differing samples at the same time at a rate of 40 revolutions per minute or more, the preferred dryer utilizes a rotational movement of 47 revolutions per minute.

Embodiments of the present invention also utilize airflow at a rate of 500 cubic feet per minute or more, in addition to the heated air and rotational movement, to create airflow within the porous containers/bags holding the feedstuff samples. The dryer of the preferred embodiment creates an airflow rate of approximately 600 cubic feet per minute. The rotational movement of the dryer will also exert one or more forces on the porous container. Accordingly, the feedstuff within may dry slightly faster due to the greater air flow on exposed sample surfaces.

The present invention decreases the drying time needed to prepare feedstuff samples by forcing heated air throughout the samples and simultaneously utilizing high velocity airflow and rotational movement along a horizontal axis to continually move air and to tumble the feedstuff samples. Additionally, heating air and forcing it through a porous container, a bag in the preferred embodiment, at higher velocities allows heat to reach all areas of feedstuff samples within the container and generally more evenly spread heat and airflow among the samples. In the preferred embodiment, the dryer air temperature is set to 60 degrees Celsius and rotates at 47 revolutions per minute with an airflow rate of 600 cubic feet per minute. In the preferred embodiment of the method, the dryer is operated for approximately 45 to 180 minutes to achieve approximately 10% or less of moisture content within feedstuff samples in porous enclosures placed in the dryer.

### BRIEF DESCRIPTION OF THE DRAWINGS

FIG. 1 is a flow chart of a first feedstuff sample drying process according to art embodiment of the method of the present invention.

FIG. 2 is a flow chart of a second feedstuff sample drying process according to an embodiment of the method of the present invention.

FIG. 3 is a perspective view of the fumbling dryer of the preferred embodiment of the method of the present invention.

FIG. 4 is a perspective view of a bag with feedstuff samples for use with the tumbling drying process of the present invention.

FIG. 4A is a perspective view of an alternative embodiment of a bag for holding feedstuff samples for use with the tumbling drying process of the present

FIG. 4B is perspective view of an alternative embodiment of a bag for Holding feedstuff samples for use with the tumbling drying process of the present invention.

FIG. 5 is a flow chart of an exemplary feedstuff sample drying process according to the preferred embodiment of the method of the present invention.

FIG. 6 is a bar graph depicting drying results of 230-gram corn silage samples dried using the process according to the preferred embodiment of the method of the present invention compared to drying results of 236-gram corn silage samples using a previous method.

FIG. 7 is a bar graph depicting drying results of 230-gram haylage samples dried using the process according to the preferred embodiment of the method of the present invention compared to drying results of 230-gram haylage samples using a previous method.

FIG. 8 is a bar graph depicting results of moisture levels of the bag according to the preferred embodiment of the method of the present invention and laboratory method moisture levels for multiple sample types.

### DETAILED DESCRIPTION

The following is a detailed description of embodiments of a feedstuff sample drying process. For ease of discussion and understanding, methods 100, 110, and 120 consistent with the process may be described with respect to certain machinery. It will be understood by one skilled in the art that the steps of the disclosed methods 100, 110, and 120 may be carried out by machinery or processes other than those specifically disclosed herein to obtain a similar or identical result. Accordingly, the following detailed description and associated figures should not be read as limiting.

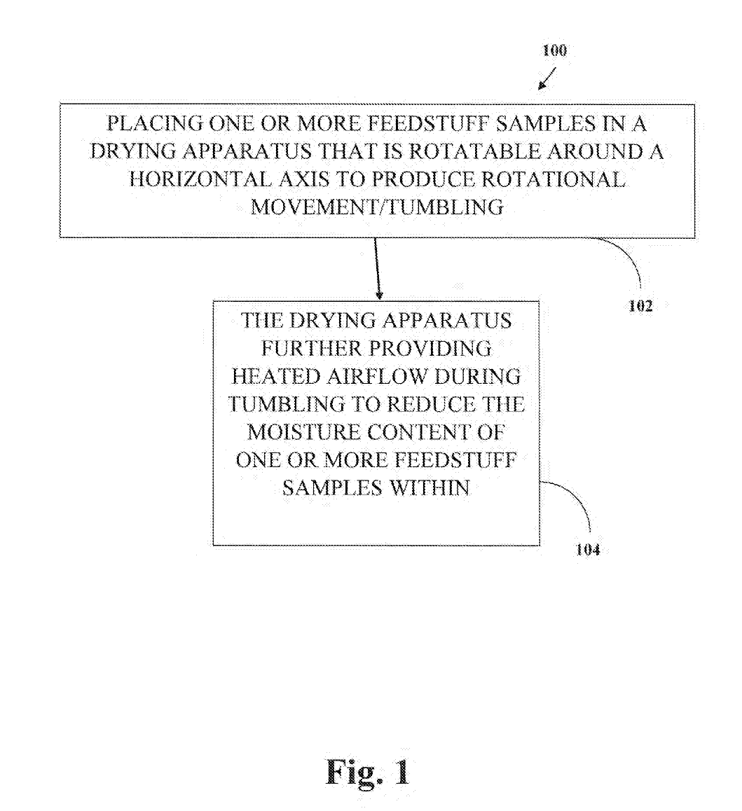

A method of feedstuff sample drying process is provided. With reference to FIG. 1, a method of the present invention 100 includes placing one or more feedstuff samples 101 in a drying apparatus 103, as provided in block 102. Furthermore, the feedstuff samples 101 within the drying apparatus 103 are subjected to the following: heated airflow and rotational movement, as shown in block 104, to reduce the moisture content of the feedstuff samples 101. The drying apparatus 103 in the preferred embodiment is a commercial grade tumbling dryer with an interior drum 103 that provides rotational movement along a horizontal axis in order to tumble the contents within the dryer 103. It should be appreciated by one skilled in the art that other devices may be utilized that provide sufficient heat, airflow and rotational movement to dry feedstuff samples to less than 10% moisture within approximately 3 hours or less without departing from the scope of the present invention. It should be noted that all temperatures provided herein are contemplated to be temperatures measured at the interior drum of the dryer

Additionally, the rotational movement, which may sometimes be referred to as tumbling, of the drying apparatus 103 will induce forces on the feedstuff samples 101 within. The forces may include, but are not limited to impacts, vibrations, centrifugal force, turbulent force, laminar stress and combinations thereof. These forces may aide the drying process by increasing the surface area of the samples 101 exposed to the heated air, dynamic airflow and/or rotational movement. The heated airflow and rotational movement constantly keep the samples 101 moving and allow the heated air to better penetrate the feedstuff samples 101.

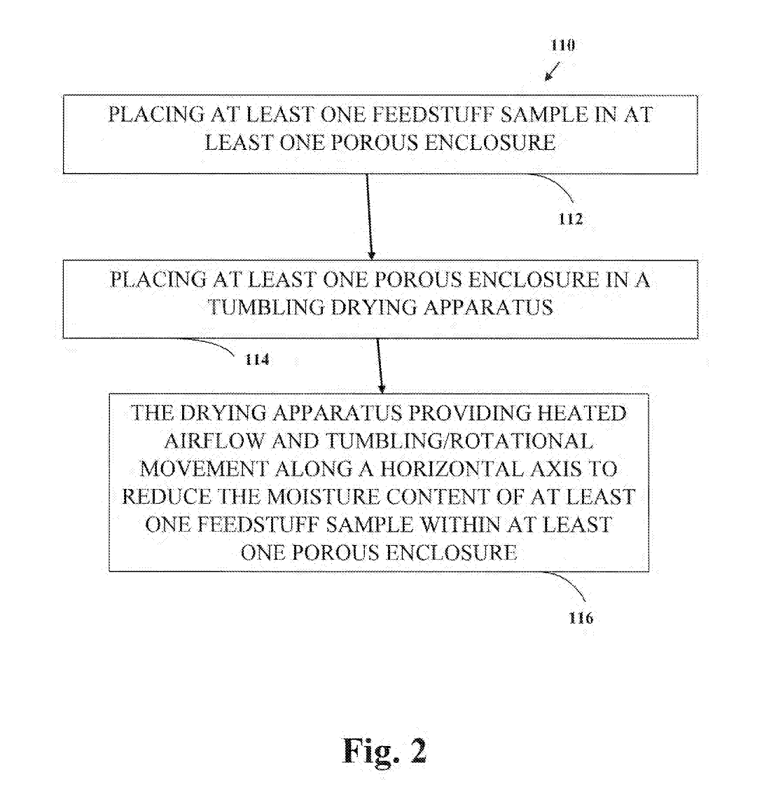

The feedstuff samples 101 used in the described method can include, but, are not necessarily limited, to, hays, fermented silage, non-fermented silage, pasture, total mixed rations, green chops, other plant tissues, shell corn, high moisture shell corn, oats, barley, wheat milo, grain mixes, feeds, byproducts, wet distillers, soybean meal, whole Bean meal, raw soybeans, other grain types and combinations thereof. It should be appreciated by one skilled in the art that any type of feedstuff samples 101 that requires drying may be processed utilizing the method of the present invention. Referring to FIG. 2, a method 110 of the feedstuff drying process of the present invention is shown. As provided in block 112, the method begins by utilizing a porous container 105 to hold one or more feedstuff samples 101. The porous enclosure 105 can be of any shape and material that may adequately hold the desired feedstuff samples 101.

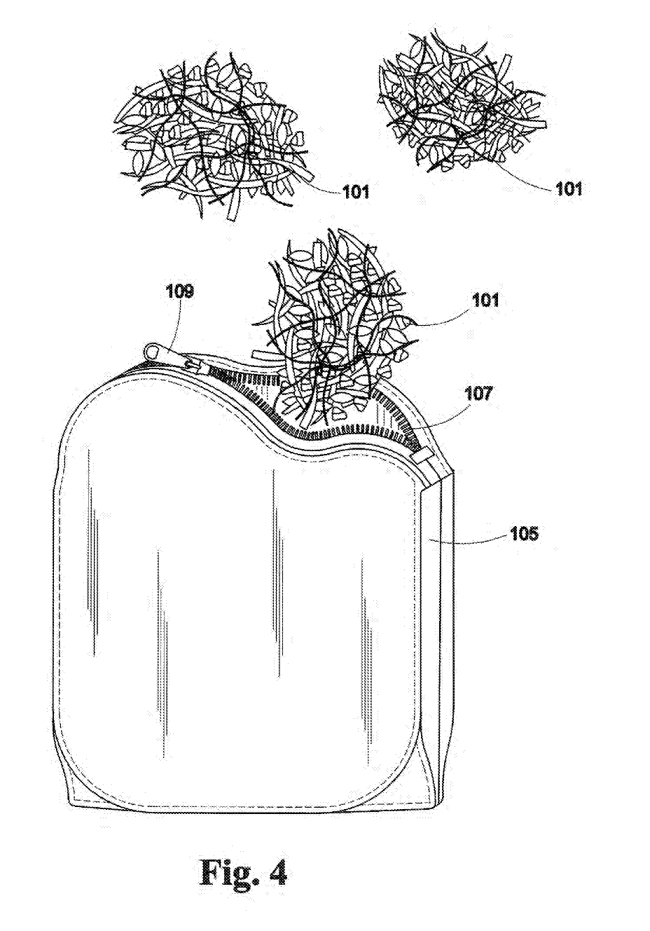

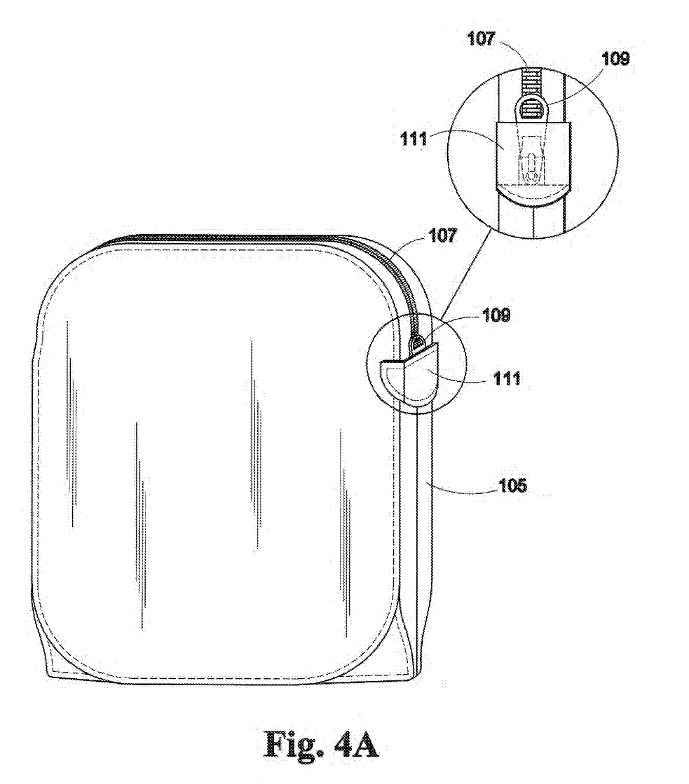

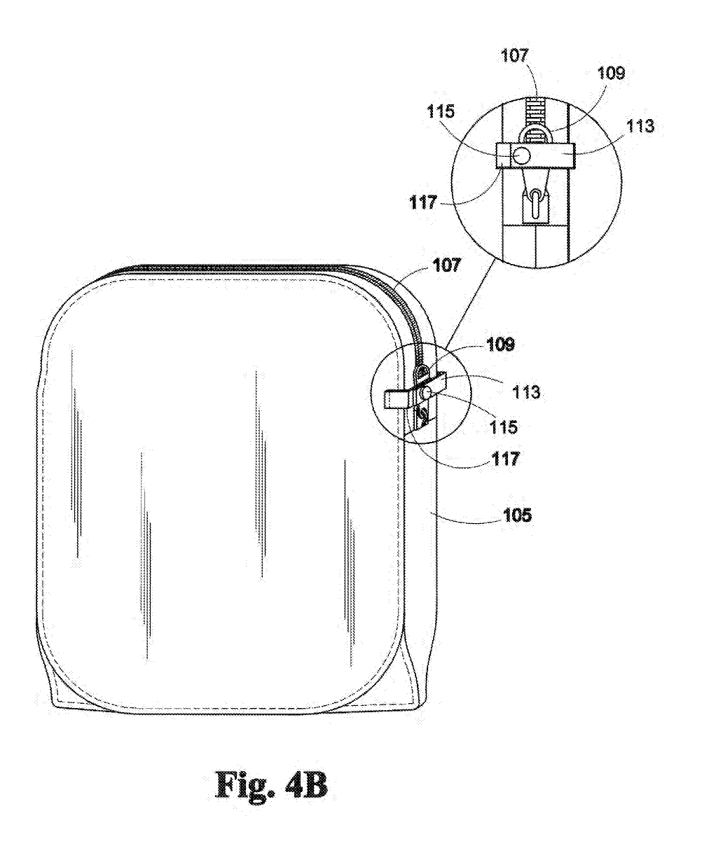

The porous enclosure 105 of the preferred embodiment is a bag with dimensions of approximately 12 inches by 16 inches with a zippered closure to retain the feedstuff samples 101 during the provided process. The hag 105 of the preferred embodiment is large enough to allow enough space so the sample(s) 101 within has adequate room to tumble back and forth freely exposing all contents to the airflow from the tumbling dryer 103. Moreover, the porous bag container 105 of the preferred embodiment utilizes materials which allow adequate airflow through the bag 105 material to allow the airflow to reach the feedstuff samples 101 within while still retaining the feedstuff samples 101 including most particulate pieces of same. The preferred embodiment of the bag 105 utilizes material with pores of approximately 20 microns in size and may be made of one or more of the following materials: cotton, polyester, spandex, nylon, muslin, broad-weave, anti-static polyester, wood pulp and combinations thereof. Moreover, the porous bag 105 of the preferred embodiment utilizes a zippered closure, with a zipper pull retention mechanism (See FIGS. 4A and 4B), to retain the feedstuff sample during the provided process. It should be understood by one skilled in the art that a porous enclosure 105 of any material, of any size, comprising any pore sizes and having any closure type adequate to hold and retain feedstuff samples 101 while allowing airflow to pass through and is also to withstand the heat and forces generated by rotational movement of the drying apparatus may be substituted without departing from the scope of the present invention.

As provided by block 114, at least one porous container 105 with at least one feedstuff sample 101 therein is then placed in a drying apparatus 103. More specifically, the drying apparatus 103 may be any device that provides adequate airflow, air temperature and/or rotational movement; such as a commercial grade tumbling dryer. As shown in block 116, the method also requires subjecting the feedstuff samples 101, placed in at least one porous container 105 within a drying apparatus 103, to heated airflow and rotational movement within the drying apparatus 103.

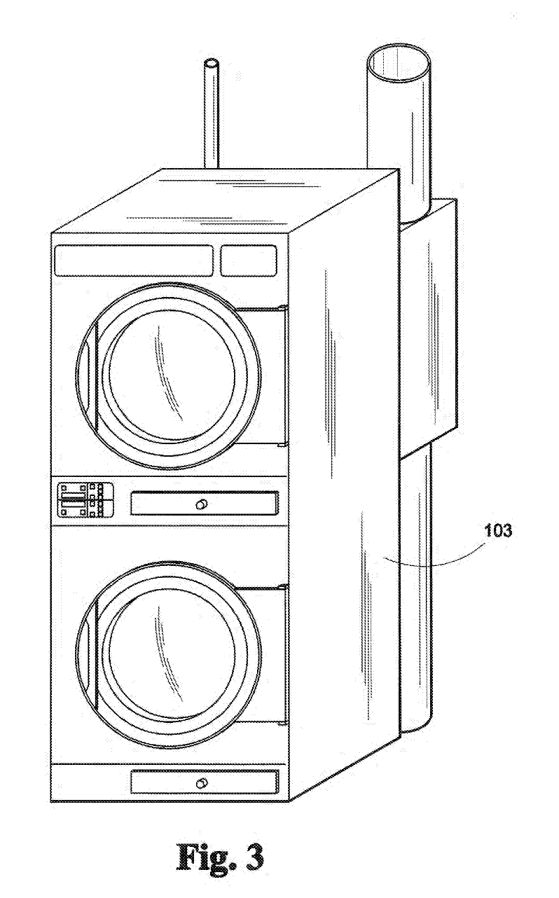

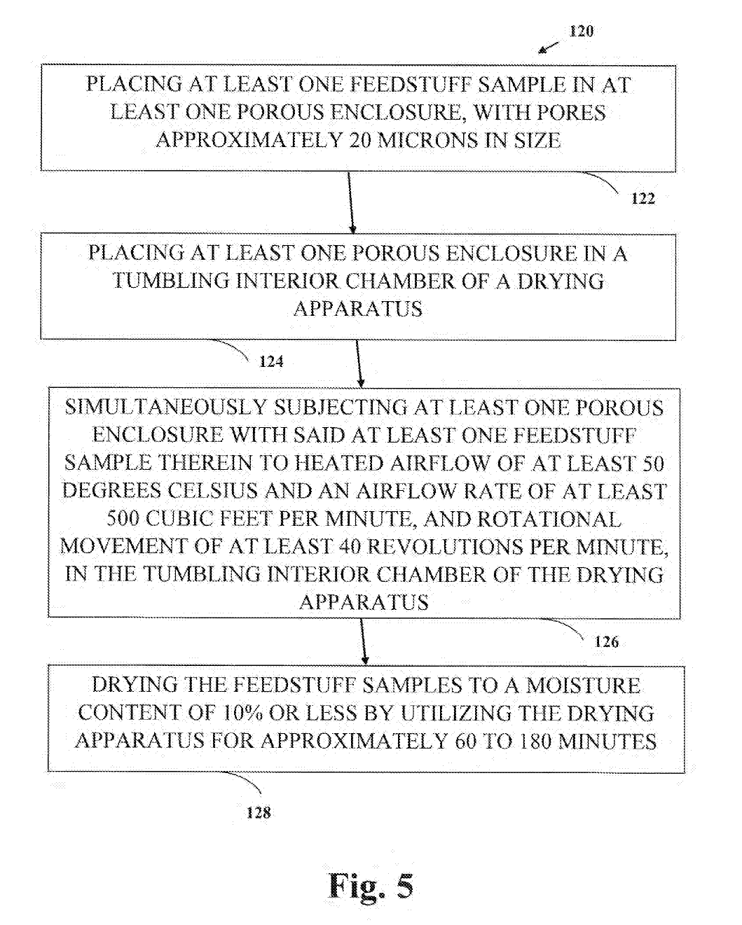

Referring to FIG. 3, the preferred embodiment of the drying apparatus 103 of the process of the present invention is shown. The drying apparatus 103 of the preferred embodiment of the process of the present invention comprises a T-30x2 Stack Dexter OPL commercial-grade, tumbling laundry dryer, hereinafter referred to as the tumbling dryer 103, to provide approximately 600 cubic feet per minute of airflow, approximately 60 degrees Celsius air temperature, and approximately 47 rotations per minute of its drum (See FIG. 5). Additionally, each unit of the tumbling dryer 103 has a capacity of 11.25 cubic feet allowing for large samples/multiple-bags of samples 101/103 to be dried simultaneously. Utilizing the preferred embodiment of the present invention, typically 40-50 bags 105 of half pound feedstuff samples 101 of varying types are placed concurrently within the tumbling dryer 103 of the present invention. It should be appreciated by one skilled in the art that other drying apparatuses may be utilized that provide the required heated airflow and rotational movement without departing from the scope of the present invention. The tumbling dryer 103 may then run for a predetermined amount of time to reduce the moisture content within the feedstuff samples 101 to the desired moisture level, typically 10% or less in the preferred embodiment. It should be appreciated by one skilled in the art that the resulting moisture level may be any amount desired based on the amount of time feedstuff samples 101 are subjected to the drying process as well as the initial moisture level of the feedstuff samples 101 without departing from the scope of the present invention.

The tumbling dryer 103 of the preferred embodiment, as depicted in FIG. 3, and described above, utilizes the same principals used by a clothes dryer to dry clothes on the feedstuff samples 101 placed in one or more porous containers 105 of the provided process. Specifically, air is brought into the tumbling dryer 103 and heated to a specified temperature. Thereafter the heated air is brought into an interior holding, chamber of the tumbling dryer 103 that holds the feedstuff samples 101 and/or porous containers 103 with the feedstuff samples therein 101 to be dried. Additionally, the tumbling dryer 103 rotates the interior holding chamber along, a horizontal axis to turn the items placed within said interior holding chamber, this is typically referred to as tumbling the items within the interior holding chamber. Simultaneously, air is pulled from the interior holding chamber using at least one fan to exhaust condensation and steam from the drying apparatus 103. As a result of the fan pulling air out of the interior holding chamber the. incoming heated, air rushes in to fill the exhausted air s volume, thereby creating the desired cubic feet per minute airflow.

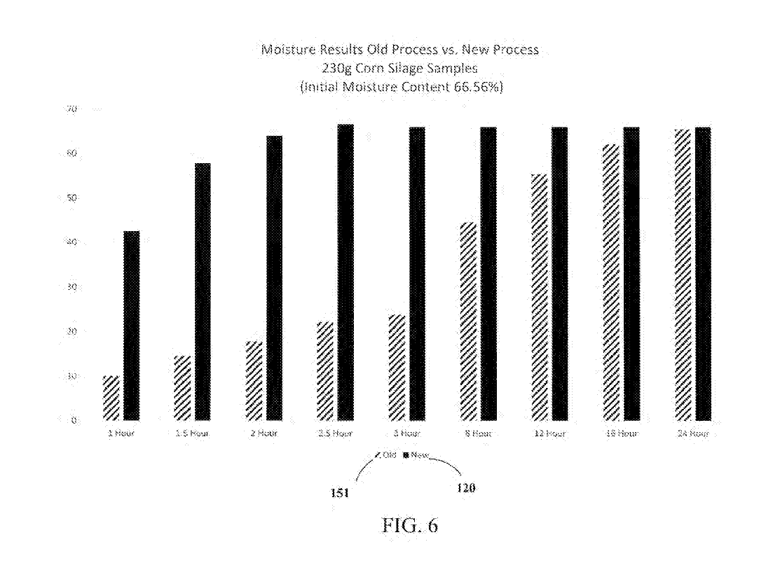

The preferred embodiment of the present invention provides a typical time of reducing feedstuff sample moisture levels to about 10% or less in approximately 3 hour or less. As depicted in FIG. 6, the difference in efficiency of moisture removal is apparent compared to previous methods. As shown, the new process 120, which is the preferred embodiment of the present invention, can remove 42.55% of the moisture in a 230-gram corn, silage feedstuff sample 101, with an original moisture content of 66.56%, in 1 hour. This is differentiated from the old method 151 which was only able to remove 10.04% moisture within the same timeframe as provided in FIG. 6. The old method 151 utilized forced air dryers with a gas furnace and blower as the heat and airflow source. The samples in the old method 151 piled the corn silage samples in metal tins that were then stacked upon one another in carts and placed inside a 224 cubic foot chamber connected to the gas furnace with blower to heat and circulate air within the chamber. The old method 151 does allow for some airflow from the blowers and the attached furnace however said airflow is minimal when compared to the new process 120.

Provided below is a first data table of 30 samples, comprising 350 grams of corn silage, in separate collection vessels. The first table shows not only moisture content of the previously described, forced air gas furnace and blower old method 151, but also shows compositional makeup of the resulting samples from both the old method 151, as described above, and the new method 120 of the current invention. All values were ascertained using near-infrared and/or x-ray analysis.

Corn Silage Study using same lot for all testing had a moisture content of approximately 67%
All Values on a 100% DM Basis except As Anaylzed Moisture (AAMST)
All Values acquired from NIR/Xray Instrumentation
350 grams weighed into each collection vessel panned and dried within same run for 10 H

| SAMPLE | Normal Prep | DESCRIPTION | AAMST | ADF | NDF | CP |
|---|---|---|---|---|---|---|
| 1161800 | Wet Rep 1 | CORN SILAGE 1 WET | 4.42 | 28.84 | 48.07 | 5.36 |
| 1161801 | Wet Rep 2 | CORN SILAGE 2 WET | 4.20 | 28.95 | 48.28 | 5.29 |
| 1161802 | Wet Rep 3 | CORN SILAGE 3 WET | 4.11 | 29.73 | 49.86 | 5.39 |
| 1161803 | Wet Rep 4 | CORN SILAGE 4 WET | 4.16 | 28.33 | 47.37 | 5.72 |
| 1161804 | Wet Rep 5 | CORN SILAGE 5 WET | 4.18 | 27.80 | 46.14 | 5.59 |
| 1161805 | Wet Rep 6 | CORN SILAGE 6 WET | 4.45 | 25.75 | 43.43 | 5.61 |
| 1161806 | Wet Rep 7 | CORN SILAGE 7 WET | 4.31 | 28.27 | 47.19 | 5.64 |
| 1161807 | Wet Rep 8 | CORN SILAGE 8 WET | 4.10 | 27.79 | 46.04 | 5.38 |
| 1161808 | Wet Rep 9 | CORN SILAGE 9 WET | 4.37 | 27.91 | 46.44 | 5.49 |
| 1161809 | Wet Rep 10 | CORN SILAGE 10 WET | 4.42 | 28.66 | 47.89 | 5.28 |
| | Old Dry method | Avg. | 4.27 | 28.20 | 47.07 | 5.48 |
| | Bag Dry Method | Avg. | 4.75 | 26.85 | 45.23 | 5.57 |
| | Old Dry method | 1-Std. Dev. | 0.14 | 1.05 | 1.72 | 0.16 |
| | Bag Dry Method | 1-Std. Dev. | 0.17 | 0.85 | 1.17 | 0.26 |
| | Old Dry method | CV | 3.20 | 3.73 | 3.65 | 2.86 |
| | Bag Dry Method | CV | 3.65 | 3.15 | 2.58 | 4.65 |

350 grams weighed into each collection vessel all 20 bags dried within same batch for 1.5 H

| 100% DM | 1 H 38 min | DESCRIPTION | AAMST | ADF | NDF | CP |
|---|---|---|---|---|---|---|
| 1162159 | Dryer Rep 1 | CORN SILAGE 1 1H36M | 4.94 | 27.33 | 45.98 | 5.58 |
| 1162160 | Dryer Rep 2 | CORN SILAGE 2 1H36M | 4.80 | 26.45 | 44.55 | 5.50 |
| 1162161 | Dryer Rep 3 | CORN SILAGE 3 1H36M | 4.72 | 27.81 | 46.38 | 5.62 |
| 1162162 | Dryer Rep 4 | CORN SILAGE 4 1H36M | 4.86 | 26.05 | 44.41 | 5.68 |
| 1162163 | Dryer Rep 5 | CORN SILAGE 5 1H36M | 4.77 | 28.79 | 44.91 | 5.92 |
| 1162164 | Dryer Rep 6 | CORN SILAGE 6 1H36M | 4.98 | 25.19 | 43.13 | 5.61 |
| 1162165 | Dryer Rep 7 | CORN SILAGE 7 1H36M | 4.42 | 28.00 | 46.89 | 5.69 |
| 1162166 | Dryer Rep 8 | CORN SILAGE 8 1H36M | 4.81 | 28.25 | 47.13 | 5.21 |
| 1162167 | Dryer Rep 9 | CORN SILAGE 9 1H36M | 4.75 | 26.72 | 44.70 | 5.87 |
| 1162168 | Dryer Rep 10 | CORN SILAGE 10 1H36M | 4.55 | 27.51 | 46.27 | 5.63 |
| 1162169 | Dryer Rep 11 | CORN SILAGE 11 1H36M | 4.84 | 26.77 | 45.21 | 5.58 |
| 1162170 | Dryer Rep 12 | CORN SILAGE 12 1H36M | 4.70 | 28.48 | 44.86 | 5.86 |
| 1162171 | Dryer Rep 13 | CORN SILAGE 13 1H36M | 4.40 | 26.34 | 44.09 | 5.80 |
| 1162172 | Dryer Rep 14 | CORN SILAGE 14 1H36M | 4.92 | 26.54 | 45.05 | 5.87 |
| 1162173 | Dryer Rep 15 | CORN SILAGE 15 1H36M | 4.75 | 27.35 | 45.87 | 5.58 |
| 1162174 | Dryer Rep 16 | CORN SILAGE 16 1H36M | 4.45 | 28.41 | 47.49 | 5.38 |
| 1162175 | Dryer Rep 17 | CORN SILAGE 17 1H36M | 4.76 | 26.36 | 44.93 | 5.39 |
| 1162176 | Dryer Rep 18 | CORN SILAGE 18 1H36M | 4.97 | 26.09 | 44.33 | 5.53 |
| 1162177 | Dryer Rep 19 | CORN SILAGE 19 1H36M | 4.86 | 25.78 | 43.63 | 5.52 |
| 1162178 | Dryer Rep 20 | CORN SILAGE 20 1H36M | 4.78 | 26.76 | 44.80 | 4.75 |

| ADIP | SP | NDIP | ASH | OIL | STARCH | LIG | IVDMD | CA |
|---|---|---|---|---|---|---|---|---|
| 0.34 | 50.59 | 0.58 | 3.34 | 2.45 | 28.93 | 3.09 | 67.27 | 0.18 |
| 0.33 | 54.04 | 0.49 | 3.28 | 2.34 | 28.70 | 3.02 | 67.12 | 0.17 |
| 0.37 | 51.64 | 0.58 | 3.12 | 2.39 | 27.49 | 3.09 | 67.19 | 0.18 |
| 0.36 | 51.83 | 0.59 | 3.36 | 2.52 | 29.31 | 2.86 | 89.09 | 0.18 |
| 0.31 | 50.37 | 0.54 | 3.43 | 2.60 | 30.28 | 2.85 | 88.97 | 0.19 |
| 0.25 | 50.56 | 0.46 | 3.22 | 2.71 | 32.76 | 2.55 | 71.15 | 0.18 |
| 0.30 | 50.74 | 0.60 | 3.71 | 2.51 | 28.52 | 2.87 | 68.61 | 0.19 |
| 0.33 | 51.55 | 0.55 | 3.53 | 2.50 | 30.53 | 2.92 | 68.30 | 0.17 |
| 0.34 | 51.62 | 0.53 | 3.58 | 2.49 | 30.13 | 2.90 | 69.02 | 0.18 |
| 0.30 | 51.09 | 0.54 | 3.53 | 2.29 | 28.18 | 2.99 | 67.36 | 0.18 |
| 0.32 | 51.40 | 0.55 | 3.41 | 2.48 | 29.48 | 2.91 | 68.41 | 0.18 |
| 0.33 | 48.87 | 0.56 | 3.51 | 2.59 | 31.44 | 2.75 | 70.75 | 0.18 |
| 0.03 | 1.07 | 0.04 | 0.18 | 0.12 | 1.51 | 0.16 | 1.26 | 0.01 |
| 0.03 | 1.41 | 0.05 | 0.22 | 0.10 | 1.12 | 0.16 | 1.28 | 0.01 |
| 10.73 | 2.07 | 8.20 | 5.29 | 4.94 | 5.11 | 5.37 | 1.84 | 3.70 |
| 7.92 | 2.88 | 9.68 | 6.14 | 3.82 | 3.57 | 5.73 | 1.80 | 5.69 |
| 0.32 | 48.68 | 0.59 | 3.84 | 2.47 | 30.50 | 2.69 | 70.69 | 0.19 |
| 0.31 | 50.00 | 0.55 | 3.61 | 2.57 | 32.19 | 2.69 | 70.56 | 0.17 |
| 0.35 | 47.86 | 0.61 | 3.79 | 2.70 | 30.55 | 2.82 | 70.46 | 0.18 |
| 0.31 | 47.41 | 0.56 | 3.46 | 2.68 | 32.07 | 2.61 | 71.59 | 0.19 |
| 0.36 | 46.63 | 0.65 | 3.59 | 2.57 | 31.79 | 2.75 | 71.52 | 0.17 |
| 0.30 | 47.84 | 0.52 | 3.65 | 2.65 | 33.73 | 2.39 | 73.24 | 0.18 |
| 0.37 | 48.71 | 0.83 | 3.66 | 2.60 | 29.68 | 2.96 | 70.06 | 0.18 |
| 0.35 | 51.21 | 0.50 | 3.62 | 2.47 | 30.05 | 3.05 | 68.26 | 0.17 |
| 0.37 | 48.12 | 0.69 | 3.77 | 2.63 | 32.03 | 2.68 | 72.04 | 0.19 |
| 0.33 | 47.49 | 0.62 | 3.46 | 2.64 | 30.44 | 2.88 | 69.77 | 0.18 |
| 0.32 | 49.72 | 0.54 | 3.42 | 2.65 | 31.05 | 2.70 | 71.40 | 0.17 |
| 0.36 | 47.13 | 0.65 | 3.41 | 2.76 | 32.10 | 2.73 | 71.52 | 0.18 |
| 0.31 | 48.01 | 0.53 | 3.24 | 2.68 | 32.13 | 2.75 | 71.54 | 0.18 |
| 0.31 | 49.72 | 0.56 | 3.41 | 2.52 | 31.37 | 2.71 | 70.80 | 0.17 |
| 0.32 | 50.09 | 0.56 | 3.65 | 2.56 | 30.57 | 2.76 | 70.39 | 0.18 |
| 0.34 | 51.75 | 0.49 | 3.45 | 2.34 | 29.36 | 3.08 | 68.15 | 0.17 |
| 0.27 | 48.73 | 0.52 | 3.02 | 2.53 | 32.51 | 2.79 | 69.67 | 0.16 |
| 0.32 | 49.71 | 0.54 | 3.27 | 2.64 | 32.23 | 2.69 | 71.86 | 0.17 |
| 0.31 | 50.48 | 0.56 | 3.66 | 2.61 | 32.69 | 2.58 | 71.93 | 0.19 |
| 0.34 | 48.36 | 0.46 | 3.18 | 2.67 | 31.68 | 2.76 | 69.48 | 0.16 |

| PHOS | MG | K | NA | SUL | CL | FE | CU | ZN |
|---|---|---|---|---|---|---|---|---|
| 0.26 | 0.16 | 1.06 | 0.01 | 0.06 | 0.13 | 101 | 3 | 23 |
| 0.25 | 0.15 | 1.06 | 0.01 | 0.06 | 0.13 | 96 | 2 | 23 |
| 0.25 | 0.15 | 1.11 | 0.01 | 0.06 | 0.14 | 98 | 3 | 23 |
| 0.26 | 0.15 | 1.09 | 0.01 | 0.06 | 0.14 | 126 | 2 | 24 |
| 0.26 | 0.16 | 1.13 | 0.01 | 0.07 | 0.14 | 136 | 4 | 23 |
| 0.26 | 0.15 | 1.06 | 0.01 | 0.06 | 0.13 | 161 | 3 | 24 |
| 0.27 | 0.16 | 1.13 | 0.01 | 0.06 | 0.14 | 206 | 3 | 26 |
| 0.24 | 0.15 | 1.05 | 0.01 | 0.06 | 0.13 | 174 | 4 | 26 |
| 0.25 | 0.15 | 1.08 | 0.01 | 0.06 | 0.14 | 198 | 2 | 23 |
| 0.25 | 0.15 | 1.11 | 0.01 | 0.06 | 0.14 | 142 | 3 | 25 |
| 0.25 | 0.15 | 1.09 | 0.01 | 0.06 | 0.14 | 144 | 3 | 24 |
| 0.24 | 0.14 | 1.06 | 0.01 | 0.06 | 0.13 | 50 | 3 | 30 |
| 0.01 | 0.00 | 0.03 | 0.00 | 0.00 | 0.01 | 40.57 | 0.74 | 1.25 |
| 0.01 | 0.01 | 0.03 | 0.00 | 0.00 | 0.00 | 4.30 | 1.11 | 4.40 |
| 3.25 | 3.16 | 2.80 | 0.00 | 5.18 | 3.80 | 28.27 | 25.44 | 5.20 |
| 3.02 | 4.01 | 2.44 | 23.54 | 6.26 | 3.41 | 8.63 | 39.46 | 14.51 |
| 0.23 | 0.14 | 1.11 | 0.01 | 0.05 | 0.13 | 55 | 4 | 31 |
| 0.24 | 0.14 | 1.06 | 0.01 | 0.08 | 0.14 | 51 | 3 | 32 |
| 0.23 | 0.13 | 1.06 | 0.01 | 0.05 | 0.13 | 50 | 4 | 30 |
| 0.25 | 0.15 | 1.10 | 0.01 | 0.06 | 0.13 | 48 | 6 | 38 |
| 0.23 | 0.13 | 1.04 | 0.01 | 0.06 | 0.13 | 53 | 2 | 32 |
| 0.24 | 0.14 | 1.04 | 0.01 | 0.06 | 0.13 | 58 | 4 | 43 |
| 0.24 | 0.14 | 1.06 | 0.01 | 0.06 | 0.13 | 49 | 3 | 30 |
| 0.24 | 0.14 | 1.07 | 0.01 | 0.06 | 0.13 | 50 | 2 | 35 |
| 0.24 | 0.14 | 1.06 | 0.01 | 0.06 | 0.13 | 58 | 3 | 33 |
| 0.24 | 0.14 | 1.05 | 0.01 | 0.06 | 0.13 | 51 | 2 | 26 |
| 0.24 | 0.14 | 1.06 | 0.01 | 0.06 | 0.13 | 49 | 3 | 29 |
| 0.24 | 0.14 | 1.05 | 0.01 | 0.06 | 0.13 | 46 | 3 | 27 |
| 0.25 | 0.15 | 1.10 | 0.01 | 0.06 | 0.13 | 52 | 2 | 28 |
| 0.25 | 0.14 | 1.07 | 0.00 | 0.06 | 0.13 | 46 | 1 | 29 |
| 0.25 | 0.14 | 1.08 | 0.01 | 0.06 | 0.13 | 60 | 2 | 26 |
| 0.24 | 0.14 | 1.04 | 0.01 | 0.06 | 0.13 | 45 | 2 | 31 |
| 0.23 | 0.13 | 1.01 | 0.01 | 0.06 | 0.12 | 43 | 2 | 27 |
| 0.24 | 0.14 | 1.05 | 0.01 | 0.06 | 0.13 | 42 | 3 | 25 |
| 0.25 | 0.15 | 1.10 | 0.01 | 0.06 | 0.14 | 52 | 3 | 28 |
| 0.23 | 0.14 | 1.03 | 0.01 | 0.05 | 0.13 | 46 | 2 | 27 |

| MN | NFC | IVTD | CWD | HEM | LACT | ACE | BUTY | PH |
|---|---|---|---|---|---|---|---|---|
| 17 | 41.37 | 73.12 | 44.08 | 19.23 | 2.92 | 2.53 | 0 | 4.0 |
| 15 | 41.30 | 73.38 | 44.88 | 19.33 | 3.08 | 2.58 | 0 | 4.1 |
| 17 | 39.82 | 72.88 | 45.61 | 20.13 | 2.89 | 2.55 | 0 | 4.1 |
| 16 | 41.63 | 74.49 | 46.15 | 19.04 | 3.11 | 2.93 | 0 | 4.0 |
| 19 | 42.78 | 74.59 | 44.93 | 18.34 | 2.79 | 2.74 | 0 | 4.0 |
| 17 | 45.49 | 76.62 | 46.17 | 17.68 | 3.26 | 2.65 | 0 | 3.8 |
| 19 | 41.55 | 74.06 | 45.03 | 18.92 | 3.31 | 2.81 | 0 | 3.8 |
| 17 | 43.10 | 74.37 | 44.33 | 18.25 | 3.15 | 2.71 | 0 | 4.0 |
| 17 | 42.53 | 74.60 | 45.31 | 18.53 | 3.11 | 2.72 | 0 | 3.9 |
| 17 | 41.55 | 73.57 | 44.81 | 19.23 | 3.38 | 2.50 | 0 | 3.9 |
| 17 | 42.11 | 74.17 | 45.13 | 18.87 | 3.10 | 2.67 | 0.00 | 3.96 |
| 17 | 43.66 | 75.89 | 46.72 | 18.36 | 3.17 | 2.56 | 0.00 | 3.86 |
| 1.20 | 1.50 | 1.07 | 0.70 | 0.69 | 0.19 | 0.14 | 0.00 | 0.11 |
| 1.15 | 1.16 | 1.17 | 1.61 | 0.37 | 0.17 | 0.12 | 0.00 | 0.09 |
| 7.00 | 3.57 | 1.44 | 1.54 | 3.66 | 6.18 | 5.11 | N/A | 2.71 |
| 6.95 | 2.67 | 1.54 | 3.45 | 2.03 | 5.24 | 4.57 | N/A | 2.20 |
| 17 | 42.72 | 75.76 | 47.28 | 18.65 | 3.26 | 2.87 | 0 | 3.8 |
| 17 | 44.31 | 75.97 | 46.06 | 18.10 | 3.21 | 2.53 | 0 | 3.8 |
| 14 | 42.13 | 75.45 | 47.07 | 18.57 | 2.83 | 2.49 | 0 | 3.8 |
| 16 | 44.33 | 76.72 | 47.58 | 18.36 | 3.22 | 2.42 | 0 | 3.9 |
| 15 | 43.65 | 76.69 | 48.10 | 18.12 | 3.11 | 2.62 | 0 | 3.9 |
| 16 | 45.48 | 78.00 | 48.99 | 17.94 | 3.20 | 2.57 | 0 | 3.8 |
| 17 | 41.89 | 74.99 | 46.66 | 18.89 | 3.06 | 2.72 | 0 | 4.0 |
| 15 | 42.07 | 73.77 | 44.35 | 18.88 | 3.00 | 2.59 | 0 | 3.8 |
| 18 | 43.67 | 77.08 | 48.68 | 17.98 | 3.15 | 2.75 | 0 | 3.8 |
| 18 | 42.60 | 75.07 | 46.12 | 18.76 | 3.10 | 2.44 | 0 | 3.9 |
| 18 | 43.68 | 76.60 | 48.24 | 18.44 | 3.38 | 2.51 | 0 | 3.8 |
| 16 | 43.77 | 76.60 | 47.84 | 18.38 | 2.93 | 2.54 | 0 | 3.9 |
| 16 | 44.73 | 76.14 | 45.88 | 17.75 | 2.99 | 2.75 | 0 | 3.9 |
| 17 | 43.91 | 76.19 | 47.15 | 18.51 | 3.48 | 2.54 | 0 | 3.9 |
| 18 | 42.88 | 75.63 | 46.87 | 18.52 | 3.15 | 2.60 | 0 | 3.9 |
| 15 | 41.83 | 73.39 | 43.97 | 19.08 | 3.15 | 2.62 | 0 | 4.1 |
| 17 | 44.65 | 74.79 | 43.89 | 18.57 | 3.18 | 2.32 | 0 | 4.0 |
| 18 | 44.77 | 77.10 | 48.34 | 18.24 | 3.44 | 2.42 | 0 | 3.9 |
| 17 | 45.15 | 77.05 | 47.40 | 17.85 | 3.36 | 2.63 | 0 | 3.8 |
| 17 | 45.06 | 74.69 | 43.95 | 18.04 | 3.25 | 2.44 | 0 | 3.8 |

| NIT | PROLA | STR7H | DIG8H | CAL | ARABO | XYLO | FRUCO | GLUCO |
|---|---|---|---|---|---|---|---|---|
| 25 | 0.52 | 96.33 | 36.13 | 568 | 2.078 | 14.089 | 0 | 0.03 |
| 23 | 0.35 | 95.88 | 37.75 | 567 | 2.117 | 14.365 | 0 | 0.05 |
| 23 | 0.40 | 94.55 | 36.51 | 569 | 2.143 | 14.715 | 0 | 0.09 |
| 24 | 0.47 | 97.28 | 39.30 | 581 | 2.097 | 14.31 | 0 | 0.15 |
| 21 | 0.61 | 98.28 | 38.55 | 581 | 2.031 | 13.939 | 0 | 0.06 |
| 26 | 0.71 | 98.25 | 39.02 | 698 | 2.102 | 13.792 | 0 | 0.06 |
| 24 | 0.57 | 97.28 | 37.11 | 576 | 2.061 | 14.02 | 0 | 0.04 |
| 24 | 0.45 | 96.83 | 38.71 | 575 | 2.035 | 13.967 | 0 | 0.10 |
| 25 | 0.56 | 97.78 | 38.25 | 579 | 2.027 | 13.934 | 0 | 0.14 |
| 21 | 0.44 | 96.21 | 36.53 | 568 | 2.079 | 14.316 | 0 | 0.05 |
| 24 | 0.51 | 96.87 | 37.69 | 576 | 2.08 | 14.14 | 0.00 | 0.08 |
| 11 | 0.73 | 96.50 | 36.86 | 592 | 2.12 | 13.73 | 0.02 | 0.07 |
| 1.65 | 0.11 | 1.16 | 1.34 | 9.65 | 0.04 | 0.28 | 0.00 | 0.04 |
| 1.81 | 0.09 | 1.07 | 1.22 | 9.06 | 0.04 | 0.19 | 0.03 | 0.07 |
| 6.98 | 21.16 | 1.20 | 3.55 | 1.68 | 1.85 | 1.96 | N/A | 54.44 |
| 17.04 | 11.60 | 1.11 | 3.31 | 1.53 | 2.03 | 1.39 | 135.59 | 92.07 |
| 11 | 0.73 | 97.16 | 36.17 | 588 | 2.11 | 14.03 | 0.04 | 0.05 |
| 13 | 0.72 | 96.95 | 36.72 | 590 | 2.11 | 13.55 | 0.04 | 0.06 |
| 12 | 0.73 | 97.47 | 35.83 | 589 | 2.14 | 13.74 | 0.00 | 0.22 |
| 11 | 0.86 | 95.30 | 35.86 | 599 | 2.15 | 13.70 | 0.00 | 0.00 |
| 8 | 0.84 | 95.68 | 37.27 | 597 | 2.09 | 13.67 | 0.00 | 0.14 |
| 12 | 0.90 | 97.71 | 38.28 | 608 | 2.09 | 13.55 | 0.07 | 0.12 |
| 6 | 0.65 | 97.18 | 38.65 | 586 | 2.19 | 13.87 | 0.00 | 0.08 |
| 8 | 0.64 | 96.81 | 34.46 | 673 | 2.08 | 13.92 | 0.00 | 0.03 |
| 10 | 0.78 | 96.58 | 37.71 | 599 | 2.11 | 13.72 | 0.04 | 0.21 |
| 10 | 0.71 | 94.74 | 35.41 | 586 | 2.15 | 13.68 | 0.00 | 0.00 |
| 11 | 0.75 | 96.00 | 37.74 | 598 | 2.16 | 13.87 | 0.00 | 0.10 |
| 10 | 0.71 | 96.81 | 37.10 | 599 | 2.16 | 13.56 | 0.04 | 0.13 |
| 10 | 0.72 | 98.70 | 39.05 | 500 | 2.14 | 13.82 | 0.00 | 0.05 |
| 12 | 0.73 | 95.71 | 37.19 | 592 | 2.13 | 13.87 | 0.00 | 0.00 |
| 12 | 0.67 | 95.68 | 36.65 | 589 | 2.12 | 13.83 | 0.00 | 0.12 |
| 9 | 0.52 | 95.94 | 35.35 | 573 | 2.20 | 14.19 | 0.00 | 0.00 |
| 13 | 0.75 | 94.91 | 35.52 | 588 | 2.12 | 13.65 | 0.08 | 0.00 |
| 11 | 0.82 | 95.24 | 37.73 | 502 | 2.14 | 13.71 | 0.04 | 0.08 |
| 12 | 0.67 | 98.14 | 38.75 | 599 | 2.12 | 13.42 | 0.05 | 0.04 |
| 12 | 0.77 | 98.57 | 37.85 | 586 | 2.00 | 13.48 | 0.00 | 0.03 |

| SUCRO | MANNOL | CYST | HIST | THREN | METH | ARG | VAL | PHENY |
|---|---|---|---|---|---|---|---|---|
| 0.07 | 1.52 | 0.070 | 0.138 | 0.204 | 0.090 | 0.158 | 0.316 | 0.225 |
| 0.03 | 1.61 | 0.068 | 0.133 | 0.189 | 0.086 | 0.153 | 0.295 | 0.123 |
| 0.02 | 1.29 | 0.068 | 0.132 | 0.188 | 0.084 | 0.158 | 0.291 | 0.208 |
| 0.01 | 0.84 | 0.071 | 0.136 | 0.198 | 0.088 | 0.159 | 0.30 | 0.223 |
| 0.07 | 1.07 | 0.073 | 0.133 | 0.202 | 0.091 | 0.162 | 0.312 | 0.231 |
| 0.03 | 1.39 | 0.079 | 0.147 | 0.212 | 0.096 | 0.166 | 0.328 | 0.247 |
| 0.05 | 1.58 | 0.071 | 0.143 | 0.210 | 0.091 | 0.163 | 0.322 | 0.235 |
| 0.05 | 1.27 | 0.073 | 0.141 | 0.204 | 0.092 | 0.161 | 0.317 | 0.232 |
| 0.06 | 1.44 | 0.072 | 0.139 | 0.203 | 0.090 | 0.153 | 0.311 | 0.028 |
| 0.11 | 1.83 | 0.065 | 0.139 | 0.197 | 0.087 | 0.155 | 0.305 | 0.219 |
| 0.06 | 1.38 | 0.071 | 0.138 | 0.201 | 0.090 | 0.159 | 0.310 | 0.226 |
| 0.08 | 1.24 | 0.078 | 0.144 | 0.205 | 0.092 | 0.170 | 0.310 | 0.234 |
| 0.03 | 0.28 | 0.00 | 0.00 | 0.01 | 0.00 | 0.00 | 0.01 | 0.01 |
| 0.02 | 0.27 | 0.00 | 0.00 | 0.00 | 0.00 | 0.01 | 0.01 | 0.01 |
| 81.97 | 20.59 | 5.74 | 3.47 | 3.99 | 3.81 | 2.49 | 3.78 | 4.98 |
| 24.07 | 21.56 | 3.63 | 2.46 | 1.86 | 1.90 | 3.70 | 1.80 | 2.33 |
| 0.10 | 1.38 | 0.074 | 0.144 | 0.206 | 0.091 | 0.17 | 0.307 | 0.232 |
| 0.09 | 1.21 | 0.075 | 0.146 | 0.206 | 0.092 | 0.188 | 0.311 | 0.234 |
| 0.10 | 0.80 | 0.075 | 0.141 | 0.207 | 0.092 | 0.176 | 0.308 | 0.234 |
| 0.07 | 1.42 | 0.080 | 0.150 | 0.212 | 0.096 | 0.178 | 0.323 | 0.245 |
| 0.07 | 0.96 | 0.080 | 0.146 | 0.210 | 0.096 | 0.181 | 0.318 | 0.244 |
| 0.10 | 1.03 | 0.080 | 0.144 | 0.203 | 0.003 | 0.174 | 0.303 | 0.237 |
| 0.08 | 1.21 | 0.075 | 0.140 | 0.208 | 0.092 | 0.173 | 0.313 | 0.234 |
| 0.11 | 1.35 | 0.071 | 0.141 | 0.201 | 0.089 | 0.162 | 0.304 | 0.224 |
| 0.07 | 0.77 | 0.079 | 0.142 | 0.203 | 0.094 | 0.175 | 0.305 | 0.236 |
| 0.06 | 1.27 | 0.075 | 0.147 | 0.205 | 0.092 | 0.173 | 0.312 | 0.233 |
| 0.06 | 1.35 | 0.075 | 0.145 | 0.199 | 0.091 | 0.169 | 0.303 | 0.229 |
| 0.08 | 0.86 | 0.080 | 0.145 | 0.207 | 0.094 | 0.179 | 0.312 | 0.239 |
| 0.09 | 1.09 | 0.078 | 0.139 | 0.209 | 0.094 | 0.169 | 0.317 | 0.239 |
| 0.06 | 1.41 | 0.078 | 0.149 | 0.207 | 0.093 | 0.170 | 0.316 | 0.237 |
| 0.08 | 1.24 | 0.075 | 0.142 | 0.204 | 0.091 | 0.170 | 0.308 | 0.232 |
| 0.04 | 1.55 | 0.071 | 0.143 | 0.209 | 0.091 | 0.185 | 0.318 | 0.230 |
| 0.08 | 1.25 | 0.075 | 0.149 | 0.204 | 0.091 | 0.164 | 0.310 | 0.229 |
| 0.07 | 1.42 | 0.077 | 0.147 | 0.199 | 0.092 | 0.168 | 0.304 | 0.230 |
| 0.08 | 1.31 | 0.077 | 0.147 | 0.204 | 0.092 | 0.185 | 0.309 | 0.234 |
| 0.05 | 1.88 | 0.073 | 0.137 | 0.198 | 0.091 | 0.155 | 0.307 | 0.225 |

| ISO | LEU | LYS | TRYP |
|---|---|---|---|
| 0.222 | 0.493 | 0.240 | 0.041 |
| 0.209 | 0.470 | 0.227 | 0.041 |
| 0.205 | 0.458 | 0.219 | 0.037 |
| 0.215 | 0.489 | 0.241 | 0.042 |
| 0.221 | 0.503 | 0.252 | 0.044 |
| 0.232 | 0.543 | 0.271 | 0.049 |
| 0.227 | 0.505 | 0.261 | 0.046 |
| 0.224 | 0.512 | 0.261 | 0.046 |
| 0.220 | 0.500 | 0.261 | 0.044 |
| 0.214 | 0.480 | 0.240 | 0.043 |
| 0.219 | 0.495 | 0.245 | 0.043 |
| 0.219 | 0.515 | 0.250 | 0.043 |
| 0.01 | 0.02 | 0.02 | 0.00 |
| 0.00 | 0.01 | 0.01 | 0.00 |
| 3.78 | 4.78 | 6.25 | 7.54 |
| 1.85 | 2.30 | 2.81 | 4.63 |
| 0.218 | 0.504 | 0.259 | 0.045 |
| 0.220 | 0.515 | 0.251 | 0.044 |
| 0.219 | 0.503 | 0.267 | 0.044 |
| 0.228 | 0.539 | 0.256 | 0.046 |
| 0.226 | 0.532 | 0.254 | 0.046 |
| 0.216 | 0.520 | 0.264 | 0.044 |
| 0.220 | 0.509 | 0.261 | 0.043 |
| 0.215 | 0.487 | 0.244 | 0.042 |
| 0.217 | 0.520 | 0.247 | 0.043 |
| 0.219 | 0.511 | 0.243 | 0.043 |
| 0.214 | 0.506 | 0.245 | 0.042 |
| 0.222 | 0.526 | 0.253 | 0.044 |
| 0.223 | 0.530 | 0.257 | 0.044 |
| 0.224 | 0.520 | 0.251 | 0.045 |
| 0.218 | 0.507 | 0.252 | 0.045 |
| 0.229 | 0.505 | 0.246 | 0.042 |
| 0.219 | 0.512 | 0.240 | 0.041 |
| 0.214 | 0.513 | 0.241 | 0.041 |
| 0.219 | 0.520 | 0.260 | 0.046 |
| 0.213 | 0.517 | 0.234 | 0.038 |

Looking to the data in FIG. 6, the new process 120 reduces the moisture level at a more rapid rate than the old process 151. Additionally, as shown in the data of FIG. 6, some of the analytical constituents, when comparing the old process versus the new process samples, may have slight Improvements. The analytical chemical composition improves as indicated by testing of soluble protein, ADF, NDF and Starch. The improvement is possibly attributed to a lack of organic matter loss from re-fermentation in crusted pans in an oven for long periods of time, often 10-12 hours, with moisture trapped within, especially on samples with greater than 40% initial moisture levels. The hazard with trapped moisture within the piled samples of the old process 151 is that it can promote microbial, enzymatic and pyrolysis reactions compromising the susceptible assays.

As illustrated in the corn-silage sample graph of FIG. 6, the new process 120, utilizing the preferred embodiment of the present invention, reduces the moisture level of 20 bags 105 of 230-gram corn silage samples 101 below 10% in approximately 1.5 hours. The resulting: samples after 1.5 hours in the process of the preferred embodiment of the present invention results in 57.76% of the moisture within the sample removed, with a 66.56% initial moisture level. Comparatively, within the same time-frame, the old process 151, as previously described, was only able to remove 14.56% of the moisture within the samples 101.

Looking to FIG. 4, the porous enclosure, a bag, 105 of the preferred embodiment of the process of the present invention is depicted. The preferred embodiment of the method provides placing at least one feedstuff sample 101 in at least one porous enclosure 105, a bag in the preferred embodiment, comprising of pores between 10 and 50 microns in size. Typically, 20-micron size pores are used with the bag 105 of the preferred embodiment. The bag 105 of the preferred embodiment is approximately 12 inches by 16 inches in size and utilizes a rectangular shape with at least two rounded corners and a zippered closure. The bag 105 is at least partially composed of breathable materials that utilizes one or more pores to allow such breathability. The pore size allows airflow, from the tumbling dryer 103 of the preferred embodiment, to flow through the bags 105 while still retaining feedstuff samples 101, including most remnant and/or particulate pieces, during the drying/tumbling process. It should be appreciated by one skilled in the art that any material, pore size, bag size, shape and bag closure that can adequately retain samples while allowing airflow to pass through can be utilized without departing from the scope of the present invention. Additionally, the bag 105 of the preferred embodiment utilizes materials that aid in reducing moisture held by said bag 105. The materials may include, but are not necessarily limited to: cotton, polyester, spandex, nylon, muslin, broad-weave, anti-static polyester, wood pulp and combinations thereof. Again, it should be appreciated by one skilled in the art that any material or combination of materials that allows for adequate airflow and drying and is able to withstand the temperature and rotational forces of the drying apparatus/tumbling dryer 103 may be used without departing from the scope of the: present invention.

Looking to FIG. 4A, depicted is an alternative embodiment of the bag 105 of the preferred embodiment of the present invention. The bag 105 depicted in FIG. 4 A provides a pocket 111 adjacent to and crossing over the closing end of the zipper 107. The zipper 107 includes a zipper pull 109 that may be placed inside the pocket 111. The insertion of the zipper pull 109 in the pocket 111 keeps the zipper pull from extraneous movements and/or damage during rotational movement of the tumbling dryer 103. Additionally, placing the zipper pull 109 inside the pocket 111 keeps the zipper pull 109 from damaging adjacent bags 165 in the tumbling dryer 103 and/or damaging the interior chamber of the tumbling dryer 103. It should be appreciated by one skilled in the art that any zipper pull retention mechanism may be utilized to keep the zipper pull 109 closer to the body of the bag 105 without departing from the scope of the present invention.

Shown in FIG. 4B is an alternative embodiment of the bag 105 of the preferred embodiment of the present invention. The bag 105 shown in FIG. 4B provides a retention mechanism 113 for holding the zipper pull 109 toward the rest of the bag 105. The retention mechanism, as depicted in FIG. 4B, includes a portion of material that protrudes from the area of the bag 105 adjacent to one side of the closing end of the zipper 107 and traverses the zipper 107. The distal end of the retention mechanism 113 includes one side of a fastening mechanism 105. On the opposite side of bag 105 adjacent to the zipper 107 from the protruding material of the retention mechanism 113 is a complimentary fastening mechanism 117, which is also integrally formed with a second section of protruding material from the first section of protruding material, to receive the fastening mechanism 115 of the retention mechanism 115. The material of the retention mechanism 115 is contemplated to be long enough to a low the fastening mechanisms 115 and 117 to engage with one another while holding the zipper pull 109 below the protruding material of the retention mechanism 113 when the bag 105 is filled with one or more feedstuff samples 101. Again, the retention of the zipper pull 109 closer to the rest of the bag 105 keeps the zipper pull 109 from extraneous movements and/or damage during rotational movement of the tumbling dryer 103. Additionally, placing the zipper pull 109 within the retention mechanism 113 keeps the zipper pull 109 from damaging adjacent bags 105 in the tumbling dryer 103 and/or damaging the interior chamber of the tumbling dryer 103. It should be appreciated by one skilled in the art that any zipper pull retention mechanism may be utilized to keep the zipper pull 109 closer to the body of the bag 105 without departing from the scope of the present invention.

Referring now to FIG. 5, an exemplary method 120 of a feedstuff drying process is shown. As provided in block 122, the method provides placing at least one feedstuff sample 101 in at least one porous enclosure 105, a bag in this preferred embodiment. As discussed above, compatible feedstuff samples can include, but are not necessarily limited to, hays, fermented silage, non-fermented silage, pasture, total mixed rations, green chops, other plant tissues, shelf corn/high moisture shell com, oats, barley, wheat, milo, grain mixes, feeds, byproducts, wet distillers, soybean meal, whole bean meal, raw soybeans, other grain types and combinations thereof. Again, it should be appreciated by one skilled in the art that any type of feedstuff sample 101, that requires drying may be processed utilizing the method of the present invention. Looking to block 124 of FIG. 5, the next step of the preferred embodiment of the provided feedstuff drying process is placing at least one porous container/bag 105 holding at least one feeds tuff sample 101 in the tumbling dryer 103. Again, the tumbling dryer 103 of the preferred method of the present invention has a capacity of 11.25 cubic feet per unit, allowing large samples and/or multiple bags 101/105 of samples to be dried simultaneously. Again, if should be appreciated by one skilled in the art that any drying apparatus with sufficient heat, airflow and rotational movement may be utilized, without departing from the scope of the present invention. The preferred embodiment of the present invention simultaneously subjects the one or more: feedstuff samples 101 in at least one porous container/bag 105 to heated airflow of at least 50 degrees Celsius, preferably 60 degrees Celsius, and at least 500 cubic, feet per minute rate of airflow, preferably 600 cubic feet per minute rate of flow, as provided in block 126. Block 126 also provides the one or more feedstuff samples 101 in at least one container/bag 105 also be subjected to rotational movement of at least 40 revolutions per minute, preferably 47 revolutions per minute, while also subjected so heated airflow as described above. It is anticipated that increasing the rate of airflow and/or rotational movement would further decrease the required drying time needed prepare feedstuff samples 101. Furthermore, it should be appreciated by one skilled lit the art that the air temperature utilized may be increased when used to dry particular types of feedstuff samples 101 that are less susceptible to compositional changes due to temperature without departing from the scope of the present invention. Conversely, it should be appreciated by one skilled in the art that the air temperature utilized may be decreased when used to dry particular types of feedstuff samples 101 that are more susceptible to compositional changes due to temperature without departing form the scope of the present invention. Running the tumbling dryer 103 containing at least one enclosure 105 of at least one feedstuff sample 101 for approximately 45 to 180 minutes will yield samples containing 10% or less moisture, as pro vided in block 128.

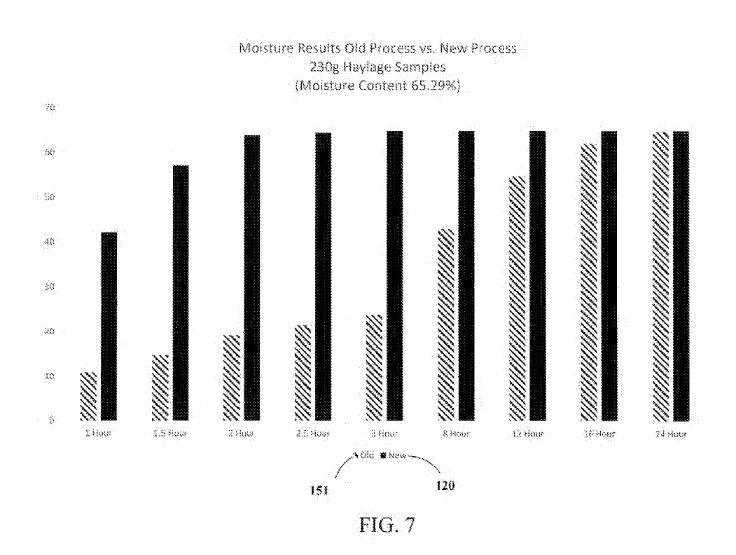

Looking again to FIG. 5, the typical time of reducing feedstuff sample moisture levels to 10% or less can be achieved in approximately 1-2.5 hours or less utilizing the process of the present invention when drying multiple bags 105, typically 40-50 bags 105, of multiple feedstuff sample types concurrently. Feedstuff samples 101 that contain approximately 70-85% initial moisture levels (wet grass silages and immature forages) may need additional time, as much as 3 hours, especially if the sample chop length exceeds 3-4 inches. Additionally, long stem samples 101 are scissor cut in the preferred embodiment of the present invention to aid in the drying process. As depicted in the graph of FIG. 7, the difference in efficiency of moisture removal is apparent even across different feedstuff types. The first graph depicted in FIG. 6 provided results for drying a 230-gram sample 101 of corn silage while the graph of FIG. 7 provides drying results for a 230-gram sample 101 of haylage. Neither graph illustrates any large differences required in the drying time of either sample type under the new process 120, which is the preferred embodiment of the present invention. As shown in FIG. 7, the new process 120 can remove 57.06% of the moisture in 230 grams of haylage feedstuff sample 101, with an original moisture content of 65.29%, in 1.5 hours. This is differentiated from the old method 151 depicted in FIG. 7 which was only able to remove 14.66% moisture within the same time frame. The old method 151 results utilized forced air dryers with a gas furnace and blower as the heat and airflow source. The samples in the old method 151 piled the haylage samples in metal tins that were then stacked upon one another in carts and placed inside a 234 cubic foot chamber connected to the gas furnace with blower to heat and circulate air within the chamber. The old method 151 does allow for some airflow from the blowers and the attached furnace however said airflow is minimal when compared to the new process 120.

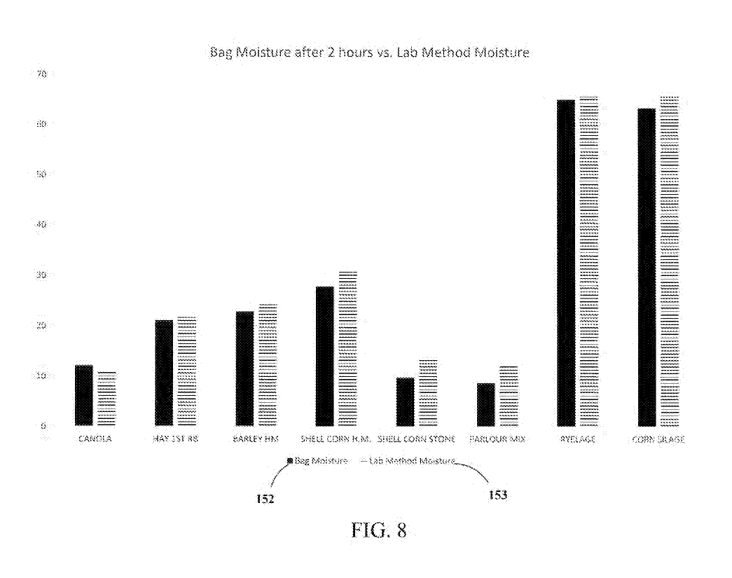

The graph of FIG. 8 provides data for moisture levels of the bag 152 of the preferred embodiment, as described above, versus laboratory method moisture levels 153 for different feedstuff sample types as listed. The samples 101 used and listed in the graph of FIG. 8 include canola, hay, high-moisture barley, high-moisture shell corn, shell corn stone, parlour mix, ryelage and corn silage. As depicted in FIG. 8, the bag moisture levels 152 after 2 hours of processing are typically below laboratory method moisture levels 153, with the exception of canola.

Additionally, provided In the second table below is a comparison of various analytical and substrate levels for various samples 101 for both the old process 151, as described above, and the new process 120, which is representative of the preferred embodiment of the present invention. The samples 101 tested with each process, with results depicted in the table below, are canola, high-moisture barley, high-moisture shell corn, hay and shell corn stone. The old process 151 data is grayed to differentiate data between tile two processes 120 and 151 tested.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g. attached, adhered, joined) are to be construed broadly and may include intermediate members between a connection Of elements and relative movement between elements. As such, joinder references do not necessarily infer that two elements are directly connected and in fixed relation to each other. In some Instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in. the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements, and/or substantial equivalents.

1. A method of drying feedstuff samples comprising:
placing at least one feedstuff sample, having an initial moisture content into a drying apparatus;
said drying apparatus rotatable around a horizontal axis; and
said drying apparatus providing heated airflow to said at least one feedstuff sample during rotation thereby adjusting the moisture content of said at least one feedstuff sample.

2. The method of claim 1 wherein said at least one feedstuff sample is placed in a porous enclosure prior to being subjected to said heated airflow and rotational movement in said drying apparatus.

3. The method of claim 1 where in the step of adjusting the moisture content of said at least one feedstuff sample results in said at least one feedstuff sample with 0-15% moisture.

4. The method of claim 1 wherein said heated airflow subjected to said at least one feedstuff sample has a temperature of at least 50 degrees Celsius.

5. The method of claim 1 wherein the step of exposing said feedstuff samples to heated airflow occurs at at least 500 cubic feet per minute.

6. The method of claim 1 wherein the step of subjecting said at least one feedstuff sample to rotational movement along a horizontal axis is produced by rotationally moving or tumbling said samples at a rate of at least 40 revolutions per minute.

7. The method of claim 1 wherein the step of adjusting the moisture content of said a t least one feedstuff sample in said drying apparatus occurs for approximately 45 to 180 minutes.

8. The method of claim 2 wherein multiple porous enclosures with said at least one feedstuff sample within can be placed in said drying apparatus concurrently.

9. A method of drying feedstuff samples comprising:
placing at least one feedstuff sample, having an initial moisture content, into at least one porous enclosure;
placing at least one said porous enclosure with said at least one feedstuff sample therein into a drying apparatus;
said drying apparatus rotatable around a horizontal axis: and
said drying apparatus providing heated airflow during rotation thereby adjusting the moisture content of said at least one feedstuff sample.

10. The method of claim 9 wherein said drying apparatus is a tumbling dryer that produces heated airflow with temperatures of at least 50 degrees Celsius, airflow of at least 500 cubic feet per minute and rotational movement of at least 40 revolutions per minute.

11. The method of claim 9 wherein said heated airflow of said drying apparatus has a temperature of 60 degrees Celsius, an airflow rate of 600 cubic feet per minute and rotational movement of 47 revolutions per minute.

12. The method of claim 9 wherein said drying apparatus provides heated airflow during rotation with said at least one porous enclosure with at least one feedstuff sample therein for approximately 45 to 180 minutes.

13. The method of claim 9 wherein said heated airflow and rotational movement of said drying apparatus results in said at least one feedstuff sample with 0-15% moisture.

14. The method of claim 9 wherein said heated airflow and rotational movement of said drying apparatus results in said at least one feedstuff sample with approximately 10% or less moisture.

15. The method of claim 9 wherein multiple porous enclosures with said at least one feedstuff sample therein are placed in said drying apparatus concurrently.

16. A method of drying feed stuff samples comprising:
placing at least one feedstuff sample, having an initial moisture content, into at least one porous enclosure with: a resealable closure mechanism;
placing at least one said porous enclosure with said at least one feedstuff sample therein into a drying apparatus;
said drying apparatus comprising a tumbling dryer with rotational movement of its interior chamber along a horizontal axis, at a rate of at least 40 revolutions per minute; and
said drying apparatus further providing heated airflow, with a temperature of at least 50 degrees Celsius and an airflow rate of at least 500 cubic feet per minute, into said interior chamber during rotation thereby adjusting the moisture content of said at least one feedstuff sample within said at least one porous enclosure.

17. The method of claim 16 wherein said drying apparatus with said at least one porous enclosure with said at least one feedstuff sample therein is subjected to heated airflow and rotational movement for approximately 45 to 180 minutes.

18. A porous enclosure for tumble drying feedstuff samples comprising:
a flexible material having a plurality of sides to form said porous enclosure to wholly contain at least one feedstuff sample;
wherein said flexible material can withstand rotational movement along an axis and the force related to said rotational movement along said axis;
said flexible material further allows airflow to pass through while retaining the material of said at least one feedstuff sample contained within said porous enclosure;
said plurality of sides of said flexible material having at least one opening to receive said at least one feedstuff sample; and
said at least one opening having a closure mechanism to retain said at least one feedstuff sample.

19. The porous enclosure of claim 18 wherein said closure mechanism is a zipper having a first side and a second side integrally formed with complimentary opposing sides of said flexible material of said at least one opening:
wherein said zipper has an originating end and receiving end;
said zipper further comprising a zipper pull attached to said first and second sides of said zipper; and
said zipper pull travels from said originating end to said receiving end to allow closure of said zipper.

20. The porous enclosure of claim 19 further comprising a secondary compartment integrally formed with said flexible material adjacent to said first or second side of said zipper and traversing said zipper to the opposite side of said opening to receive and hold said zipper pull.

21. The porous enclosure of claim 19 further comprising a first additional material integrally formed with and protruding from said flexible material adjacent to said receiving end of said first side of said zipper;
said first additional material traveling over said first arid second sides of said zipper on said receiving end to the flexible material adjacent to the second side of said zipper;
said first additional material further integrally formed with at least one end of a linking mechanism on its distal end; and
wherein a complimentary linking mechanism is integrally formed with a second additional material protruding from said flexible material adjacent to said second side of said zipper; and said first and second additional materials and linking mechanisms allows said zipper pull to be field against said porous enclosure when said zipper pull is on said receiving end and said porous enclosure is closed.
